(19) United States
(12) Patent Application Publication
(10) Pub. No.: US 2025/0114394 A1
(43) Pub. Date: Apr. 10, 2025

(54) COMPOSITIONS INCORPORATING SULFATED POLYSACCHARIDES FOR INHIBITING SARS-COV-2

(71) Applicant: RENSSELAER POLYTECHNIC INSTITUTE, Troy, NY (US)

(72) Inventors: Robert John LINHARDT, Albany, NY (US); So-Young Kim, Rockville, MD (US); Weihua Jin, Troy, NY (US); Jonathan Seth Dordick, Schenectady, NY (US); Fuming Zhang, Watervliet, NY (US); Seok-Joon Kwon, Niskayuna, NY (US); Paul S. Kwon, Niskayuna, NY (US); Keith Fraser, Troy, NY (US)

(73) Assignee: RENSSELAER POLYTECHNIC INSTITUTE, Troy, NY (US)

(21) Appl. No.: 18/597,050

(22) Filed: Mar. 6, 2024

Related U.S. Application Data
(63) Continuation-in-part of application No. 17/915,783, filed on Sep. 29, 2022.
(60) Provisional application No. 63/064,611, filed on Aug. 12, 2020.
(60) Provisional application No. 63/004,302, filed on Apr. 2, 2020.
(60) Provisional application No. 63/450,198, filed on Mar. 6, 2023.
(60) Provisional application No. 63/560,012, filed on Mar. 1, 2024.

(51) Int. Cl. A61K 31/727; A61K 31/737; A61K 45/06; A61P 31/04

(52) U.S. Cl. CPC A61K 31/727; A61K 31/737; A61K 45/06; A61P 31/04

(57) ABSTRACT

The composition inhibits severe acute respiratory syndrome coronavirus 2 (SARS-COV-2) via competitive binding to SARS-COV-2 spike protein. The composition includes a plurality of sulfated glycosaminoglycans which bind to SARS-COV-2 spike protein, preventing binding to and uptake by host cells. The sulfated glycosaminoglycans, including N-, 2-O, 3-O, or 6-O sulfate groups, or combinations thereof, include heparins and fucoidans, such as those isolated from brown seaweed. The compositions show antiviral activity, with EC₅₀ as low as 0.08 μM, and low cytotoxicity, making it promising for clinical use. While established SARS-COV-2 treatments such as remdesivir need to be administered intravenously, the compositions discussed herein are advantageously capable to being delivered as a nasal spray, metered dose inhaler, oral delivery, etc.

### CROSS REFERENCE TO RELATED APPLICATION(S)

This application is a continuation-in-part of U.S. Utility patent application Ser. No. 17/915,783, filed Sep. 29, 2022, which is a national stage patent application filing of International Application No. PCT/US2021/025500, filed Apr. 2, 2021, which claims the benefit of U.S. Provisional Application Nos. 63/064,611, filed Aug. 12, 2020, and 63/004,302, filed Apr. 2, 2020, which are incorporated by reference as if disclosed herein in their entireties. This application also claims the benefit of U.S. Provisional Patent Application Nos. 63/450,198, filed Mar. 6, 2023, and 63/560,012, filed Mar. 1, 2024, which are incorporated by reference as if disclosed herein in their entireties.

### STATEMENT REGARDING FEDERALLY SPONSORED RESEARCH AND DEVELOPMENT

This invention was made with U.S. Government support under Grant Numbers DK111958, CA231074, NS088496, and AG062344 awarded by National Institutes of Health. The United States Government has certain rights in the invention.

### BACKGROUND

In March 2020, the World Health Organization declared severe acute respiratory syndrome-related coronavirus 2 (SARS-COV-2) a pandemic less than three months after its initial emergence in Wuhan, China. SARS-COV-2 is a zoonotic Betacoronavirus transmitted through person-person contact through airborne and fecal-oral routes, and has caused nearly 125 Million confirmed coronavirus disease 2019 (COVID-19) cases and more than 2.7 million associated deaths to date worldwide.

In an effort to mitigate disease symptoms and impede viral spread, efforts in vaccine development and drug discovery are being conducted at a rapid pace. While there is limited understanding of SARS-COV-2 pathogenesis, extensive studies have been performed on how its closely related cousins, SARS-COV and MERS-COV (Middle East respiratory syndrome-related coronavirus), invade host cells. Upon initially contacting the surface of a host cell, SARS-COV and MERS-COV exploit host cell proteases to prime their surface spike glycoproteins (SGPs) for fusion activation, which is achieved by receptor binding, low pH, or both. SARS-COV and other pathogens arrive at a host cell surface by clinging, through their surface proteins, to linear, sulfated polysaccharides called glycosaminoglycans (GAGs).

The repeating disaccharide units of GAGs, comprised of a hexosamine and a uronic acid or a galactose residue, are often sulfated. GAGs are generally found covalently linked to core proteins as proteoglycans (PGs) and reside inside the cell, at the cell surface, and in the extracellular matrix (ECM). GAGs facilitate various biological processes, including cellular signaling, pathogenesis, and immunity, and possess diverse therapeutic applications. For example, an FDA approved anticoagulant heparin (HP) is a secretory GAG released from granules of mast cells during infection. Some GAG binding proteins can be identified by amino acid sequences known as Cardin-Weintraub motifs corresponding to ‘XBBXBX’ and ‘XBBBXXBX’, where X is a hydropathic residue and B is a basic residue, such as arginine and lysine, responsible for interacting with the sulfate groups present in GAGs.

### SUMMARY

Some embodiments of the present disclosure include a composition for inhibiting severe acute respiratory syndrome coronavirus 2 (SARS-COV-2) including a plurality of inhibitors configured to bind to SARS-COV-2 spike protein, wherein the at least one of the inhibitors includes a heparin or functional equivalent thereof, a fucoidan or functional equivalent thereof, or combinations thereof. In some embodiments, the inhibitors include unfractionated USP-heparin, a trisulfated (TriS) heparin, a non-anticoagulant low molecular weight heparin (NACH), or combinations thereof. In some embodiments, the inhibitors include a fucoidan, wherein the fucoidan includes the structure according to Formula I:

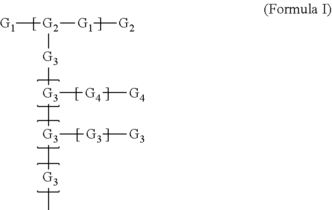

wherein G₁ is a glucuronic acid; G₂ is a mannose; G₃ is a fucose; and G₄ is a galactose. In some embodiments, a plurality of G₂, G₃, and G₄ are sulfated. In some embodiments, the fucoidan includes the structure according to Formula II:

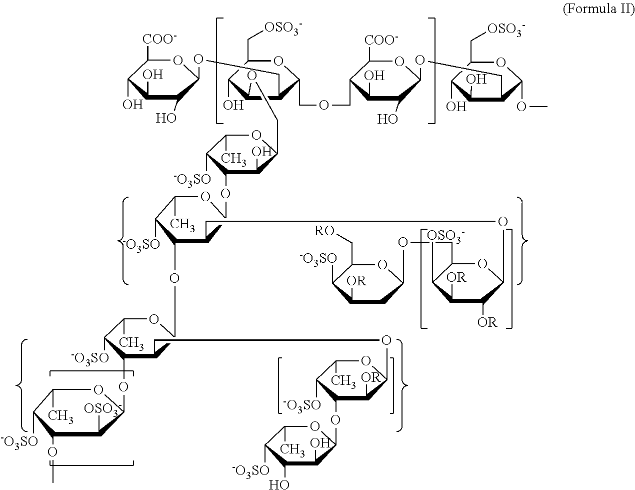

wherein each R is one of H or SO₃⁻. In some embodiments, the fucoidan has a molecular weight greater than about 10 kDa. In some embodiments, the fucoidan has a molecular weight of about 100 kDa. In some embodiments, the composition includes one or more additional active ingredients, one or more pharmaceutically acceptable adjuvants, diluents, excipients, carriers, or combinations thereof.

Some embodiments of the present disclosure include a method of inhibiting SARS-COV-2 in a patient including identifying a presence of SARS-COV-2 infection in the patient and administering an effective amount of a composition including one or more inhibitors configured to bind to SARS-COV-2 spike protein, the one or more inhibitors including a heparin or functional equivalent thereof, a fucoidan or functional equivalent thereof, or combinations thereof. In some embodiments, administering the effective amount of the composition produces a peak plasma concentration in the patient less than about 60 μM. In some embodiments, administering the effective amount of the composition produces a peak plasma concentration in the patient less than about 5 μM. In some embodiments, the composition is administered orally, nasally, pulmonary, transdermally, or combinations thereof. In some embodiments, the method includes obtaining a sample of fucoidan from seaweed and incorporating the sample of fucoidan into a composition.

Some embodiments of the present disclosure include a method of inhibiting severe acute respiratory syndrome coronavirus 2 in a patient including identifying a presence of SARS-COV-2 infection in the patient and administering an effective amount of a composition including one or more inhibitors configured to bind to SARS-COV-2 spike protein to produce a peak plasma concentration in the patient less than about 2 μM, the one or more inhibitors including the structure according to Formula I and/or Formula II.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1A is a graph showing results of surface plasmon resonance experiments used to screen polysaccharides that outcompete immobilized heparin binding to severe acute respiratory syndrome coronavirus 2 (SARS-COV-2) spike protein;

FIG. 1B show images of focus reduction assays of virus infection on treatment of indicated polysaccharides;

FIGS. 1C-1G show graphs reporting SARS-COV-2 inhibition and cellular toxicity effects of polysaccharides according to some embodiments of the present disclosure;

FIGS. 2A and 2B are charts of methods of inhibiting SARS-COV-2 in a patient according to some embodiments of the present disclosure;

FIG. 3 is a chart of a method of inhibiting SARS-COV-2 in a patient according to some embodiments of the present disclosure.

FIGS. 4A-4D are graphs showing dose response curves of inhibitory activities of fucoidan fractions, heparin, and pentosan polysulfate (PPS) on wild-type SARS-COV-2 spike glycoprotein (SGP) receptor-binding domain (RBD)-heparin surface plasmon resonance (SPR) chip binding;

FIG. 5A are images showing entry-level inhibition of wild-type SARS-COV-2;

FIG. 5B is a graph showing dose response curves against wild-type SARS-COV-2 of various sulfated polysaccharides;

FIGS. 6A-6C are graphs showing morbidity and mortality data of mice after LD₅₀ pneumonic infection with SARS-COV-2 virus;

FIGS. 7A-7B are graphs showing morbidity and mortality data of mice after 5×LD₅₀ pneumonic infection with SARS-COV-2 virus; and

FIGS. 8A-8B are graphs showing analysis of viral burden in mice after challenge with SARS-COV-2 virus.

### DESCRIPTION

Some embodiments of the present disclosure are directed to a composition for inhibiting severe acute respiratory syndrome coronavirus 2 (SARS-COV-2). In some embodiments, the composition is configured to bind to SARS-COV-2 spike glycoproteins, also referred to as “spike proteins” or “SGPs” herein. In some embodiments, the composition includes one or more inhibitors configured to bind to SARS-COV-2 spike protein. In some embodiments, the composition includes a plurality, e.g., two or more, inhibitors configured to bind to SARS-COV-2 spike protein. In some embodiments, the inhibitors include one or more glycosaminoglycans, also referred to herein as “GAGs.” In some embodiments, the one or more GAGs include a plurality of N-, 2-O, 3-O, or 6-O sulfate groups, or combinations thereof. In some embodiments, at least some of the GAGs are fully sulfated. In some embodiments, at least one of the GAGs is branched. Host cell invasion by SARS-COV and MERS-COV have been extensively studied. Without wishing to be bound by theory, upon initially contacting the surface of a host cell, SARS-COV and MERS-COV exploit host cell proteases to prime their surface SGPs for fusion activation, which is achieved by receptor binding, low pH, or both. The receptor binding domain (RBD) resides within subunit 1 (S1) while subunit 2 (S2) facilitates viral-host cell membrane fusion. Activated SGP undergoes a conformational change followed by an initiated fusion reaction with the host cell membrane. Endocytosed virions are further processed by the endosomal protease cathepsin L in the late endosome. Both MERS-COV and SARS-COV undergo proteolytic cleavage at their S2′ site, but not at their S1-S2 junction, for successful membrane fusion and host cell entry. Additionally, receptors involved in fusion activation of SARS-COV and MERS-COV include heparan sulfate (HS) and angiotensin-converting enzyme 2 (ACE2), and dipeptidyl peptidase 4 (DPP4), respectively.

While structurally similar to SARS-COV or MERS-COV SGPs, examination of the SARS-COV-2 spike protein sequence revealed GAG-binding motif resides within the S1-S2 proteolytic cleavage motif (furin cleavage motif BBXBB) that is not present in SARS-COV or MERS-COV SGPs. Additionally, GAG-binding-like motifs can be found within RBD and S2′ proteolytic cleavage site in SARS-COV-2 spike protein. Without wishing to be bound by theory, as discussed above, GAGs contribute to SARS-COV-2 fusion activation and host cell entry via spike protein binding. Thus, competitive inhibition by a concentration of extracellular GAGs can be used to prevent SGP binding to host cells and thus prevent cell fusion, entry, and cellular infection. In some embodiments, the inhibitors in the composition include a heparin, a functional equivalent of heparin, a fucoidan, a functional equivalent of fucoidan, or combinations thereof. In some embodiments, the functional equivalents of heparin and fucoidan are naturally occurring, synthetically synthesized, or combinations thereof. In some embodiments, the inhibitors include unfractionated USP-heparin, a trisulfated (TriS) heparin, a non-anticoagulant low molecular weight heparin (NACH), or combinations thereof. In some embodiments, the fucoidan includes the structure according to Formula I:

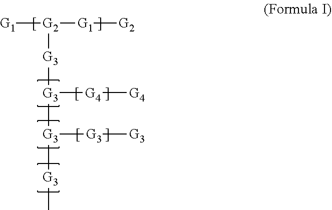

In some embodiments, G₁ is a glucuronic acid. In some embodiments, G₂ is a mannose. In some embodiments, G₃ is a fucose. In some embodiments, G₄ is a galactose. In some embodiments, a plurality of G₂, G₃, and G₄ are sulfated. In some embodiments, G₂, G₃, and G₄ are all fully sulfated. In some embodiments, the fucoidan includes the structure according to Formula II:

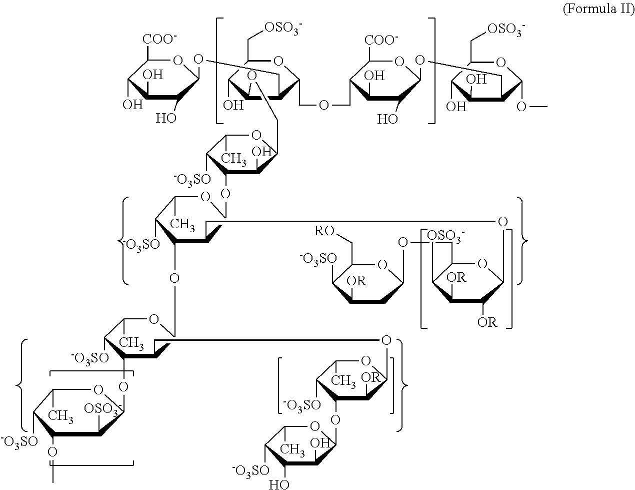

In some embodiments, each R is one of H or SO₃⁻. In some embodiments, a plurality of R is SO₃⁻. In some embodiments, each R is SO₃⁻. In some embodiments, the GAGs in the composition have an average molecular weight of about 1 kDa, 5 kDa, 10 kDa, 15 kDa, 20 kDa, 25 kDa, 30 kDa, 35 kDa, 40 kDa, 45 kDa, 50 kDa, 60 kDa, 70 kDa, 80 kDa, 90 kDa, or 100 kDa. In some embodiments, the fucoidan has a molecular weight greater than about 10 kDa. In some embodiments, the fucoidan has a molecular weight of about 100 kDa. In some embodiments, the GAGs are extracted from seaweed, e.g., Saccharina japonica. In some embodiments, the GAGs are extracted from Saccharina latissima. In some embodiments, the composition includes one or more additional active ingredients. In some embodiments, the composition includes one or more antivirals. In some embodiments, the composition includes, or is utilized in combination with, one or more therapeutics for SARS-COV-2, e.g., remdesivir. In some embodiments, the composition includes one or more pharmaceutically acceptable adjuvants, diluents, excipients, carriers, or combinations thereof. In some embodiments, the composition is included in a therapeutic for administration to a patient, e.g., orally, nasally, via inhalation, nebulization, transdermally, intravenously, or combinations thereof.

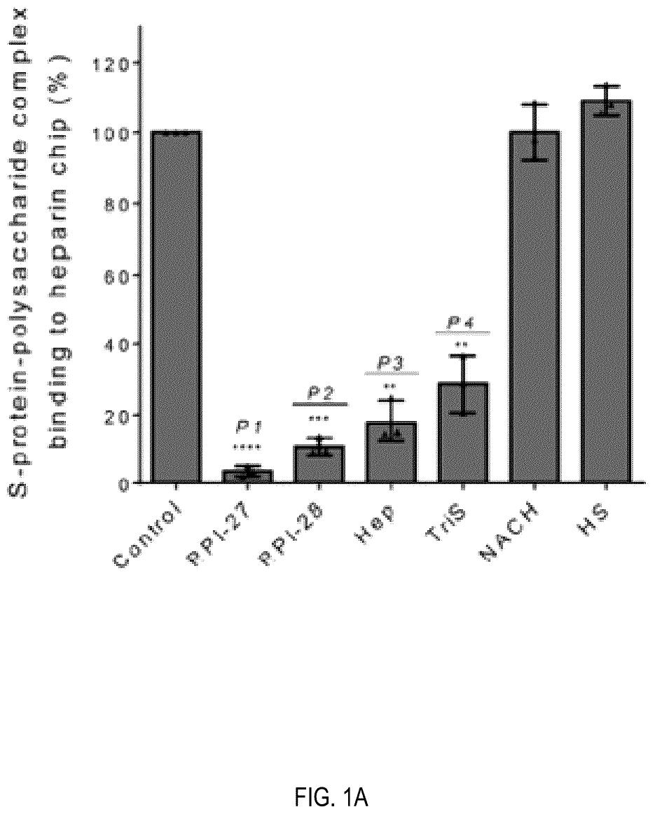

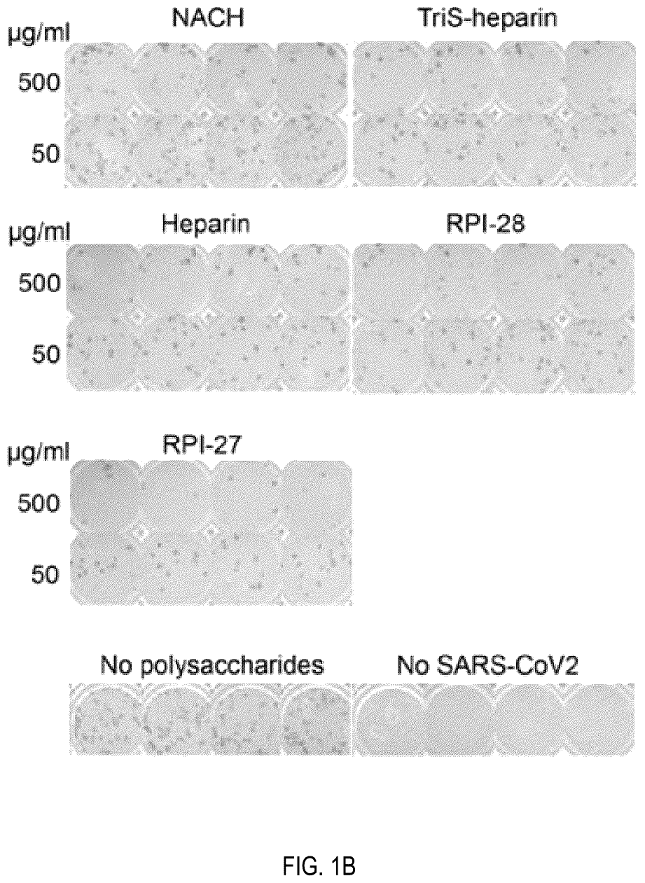

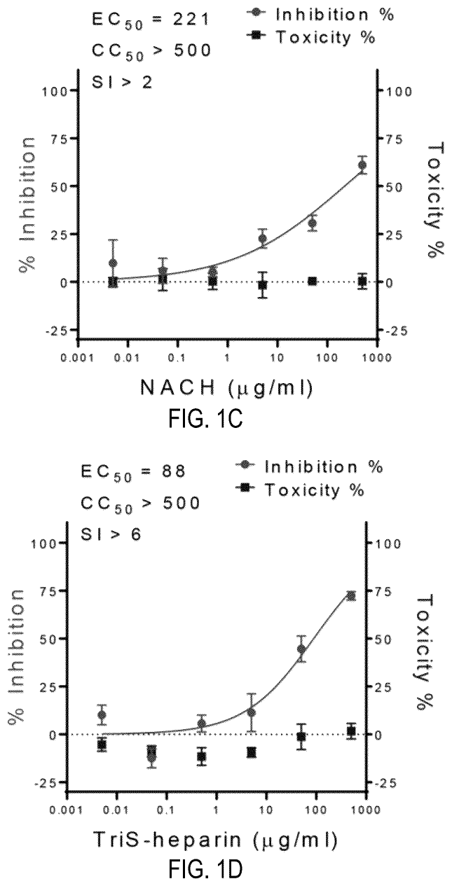

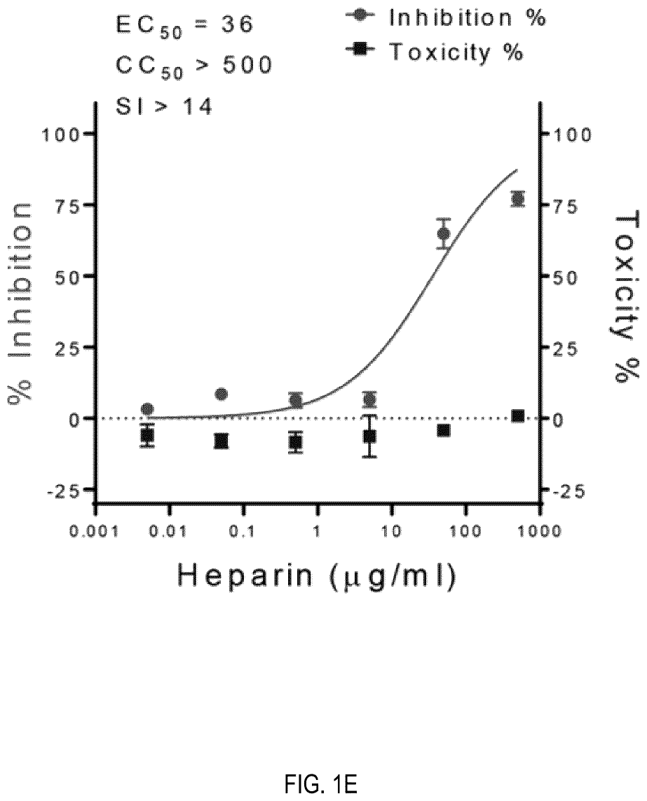

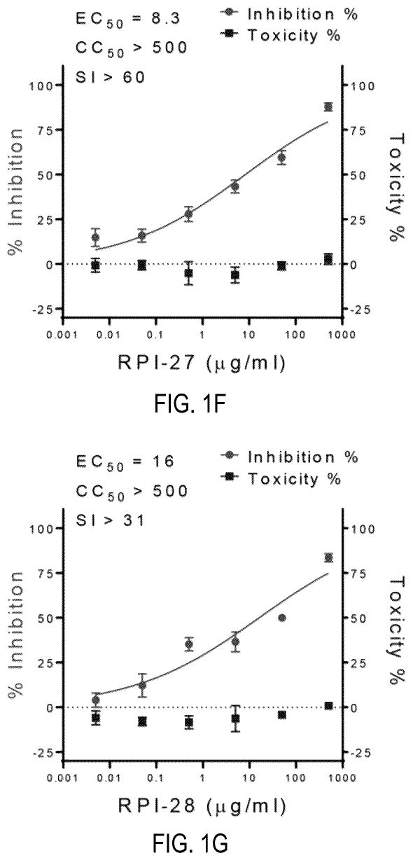

Referring now to FIG. 1A, heparin, heparan sulfates, other GAGs, and fucoidan and other highly sulfated polysaccharides were screened using surface plasmon resonance (SPR) to measure binding affinity to the SARS-COV-2 spike protein. Solution competition studies between surface immobilized heparin and other sulfated polysaccharides were evaluated by injecting SARS-COV-2 spike protein (50 nM) alone or mixed with 1 μM of an indicated polysaccharide in SPR buffer at a flow rate of 30 μL/min. After each run, dissociation and regeneration were performed. For each set of competition experiments, a control experiment (spike protein without polysaccharide) was performed to ensure the surface was fully regenerated. Among the tested polysaccharides, RPI-27 and RPI-28, complex sulfated polysaccharides (fucoidans) extracted from the seaweed Saccharina japonica, chemo-enzymatically synthesized trisulfated (TriS) heparin, and unfractionated USP-heparin itself competed with heparin for spike protein binding. These compounds along with a non-anticoagulant low molecular weight heparin (NACH) were selected for further study.

Referring now to FIG. 1B, standard assays were performed to quantify potential cytotoxicity and antiviral activity. Cytotoxicity determination of the polysaccharides was performed using Vero cells and the standard water-soluble tetrazolium salt-1 (WST-1) assay (Takara Bio Inc., Japan). None of the tested polysaccharides showed toxicity even at the highest concentrations tested. Vero cells were infected with SARS-COV-2 at a multiplicity of infection (MOI) of 2.5×10⁻³ with varying dosages of polysaccharide to confirm antiviral activity. A focus reduction assay was performed 48 h post infection to determine efficacy. Antiviral activities correlated with the SPR results.

RPI-27 is a high molecular weight, branched polysaccharide related to fucoidan, and had an EC₅₀ of 8.3±4.6 μg/mL, which corresponds to ˜83 nM (Table 1 below). This is substantially more potent than remdesivir having a reported in vitro EC₅₀ value of 770 nM in Vero-E6 cells and 11.4 μM in Vero-CCL81 cells, currently approved for emergency use for severe COVID-19 infections. The smaller RPI-28 has the same basic structure as RPI-27, but a lower molecular weight and lower activity (EC₅₀=1.2 μM, Table 1 below). USP-heparin and the TriS-heparin (an intermediate in the bioengineered heparin synthesis pathway) also have potent antiviral activity with EC₅₀ values of ˜2.1 and 5.0 μM, while the lower molecular weight NACH had an approximate EC₅₀ of 55 μM. Heparin and TriS-heparin are similar, with the latter devoid of the relatively small fraction of 3-O-sulfate groups present on heparin. However, the low molecular weight NACH had far lower antiviral activity.

TABLE 1
In vitro effects of sulfated polysaccharides
on the inhibition of SARS-CoV2 infection.

| | | | | Binding | | |
|---|---|---|---|---|---|---|
| | Mw^{a} | Anti-coagulant | | inhibition^{c} | EC₅₀ | EC₅₀^{e} |
| Poly-saccharides | (kDa) | activity^{b} | Scaffolds | (%) | (μg/mL) | (μM) |
| NACH | 4 | − | Linear | ND^{d} | 221 ± 122 | 55 |
| TriS-heparin | 17.6 | − | Linear | 72 ± 8.0 | 88 ± 27 | 5.0 |
| Heparin | 17 | + | Linear | 82 ± 7.5 | 36 ± 14 | 2.1 |
| RPI-27 | 100 | − | Branched | 97 ± 1.5 | 8.3 ± 4.6 | 0.08 |
| RPI-28 | 12 | − | Branched | 90 ± 1.7 | 16 ± 11 | 1.2 |

^{a}average molecular weight.
^{b}antithrombin III-mediated anticoagulant activity.
^{c}competitive binding inhibition between polysaccharide-spike protein complex and heparin (HP)-SPR chip.
^{d}not detected.
^{e}the values obtained by dividing average molecular weight.

Without wishing to be bound by theory, the high activity of RPI-27 and RPI-28 relative to the other polysaccharides tested may be a result of multivalent interactions between the polysaccharide and viral particle. While USP-heparin, TriS-heparin, and NACH are linear polysaccharides, RPI-27 and RPI-28 are both highly branched, possibly conferring added points of interaction in 3-dimensional space. The higher affinity of RPI-27 compared to RPI-28, and hence its more potent antiviral activity, may be due to the far higher molecular weight of the former providing greater opportunity for multipoint binding to the spike protein of SARS-COV-2. The non-anticoagulant TriS-heparin may be more desirable in some applications than the potent anticoagulant heparin.

Without wishing to be bound by theory, these results reveal that sulfated polysaccharides bind tightly to the spike protein of SARS-COV-2, and thus, they can act as decoys to interfere with spike protein binding to the heparan sulfate co-receptor in host tissues, inhibiting viral infection. To model this, a docking model between heparin and the spike protein receptor-binding site (RBD) using the crystal structure of the chimeric RBD-ACE2 complex (PDB ID:6VW1) was constructed. The RBD's amino acid residues involved in binding the ACE2 (angiotensin-converting enzyme 2) receptor also participated in heparin binding, suggesting a mechanism of viral entry inhibition by heparin. Moreover, the larger the oligosaccharide model used in docking studies, the tighter the binding. Specifically, the octasaccharide binds tighter than the tetrasaccharide (−7.3 vs. −6.1 kcal/mol).

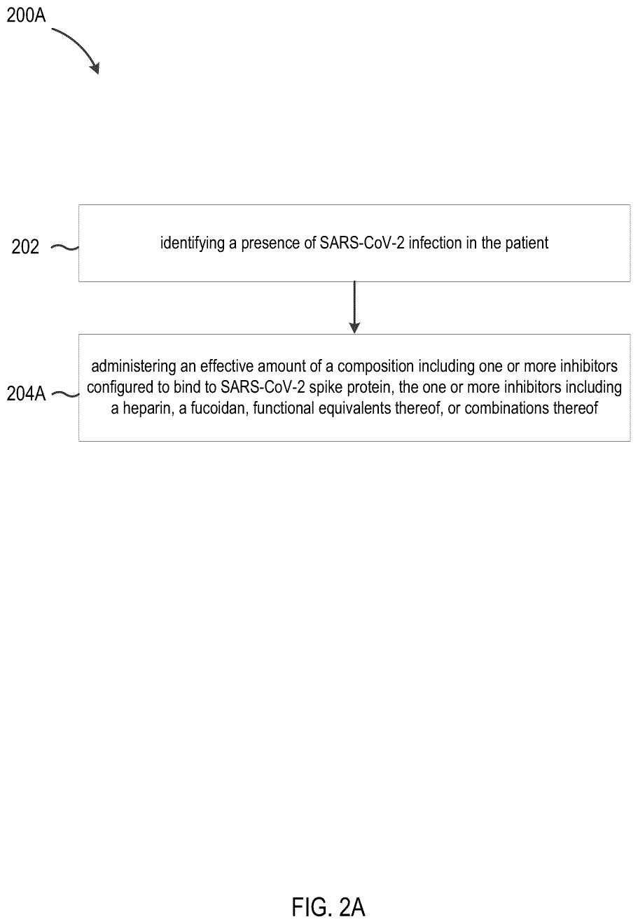

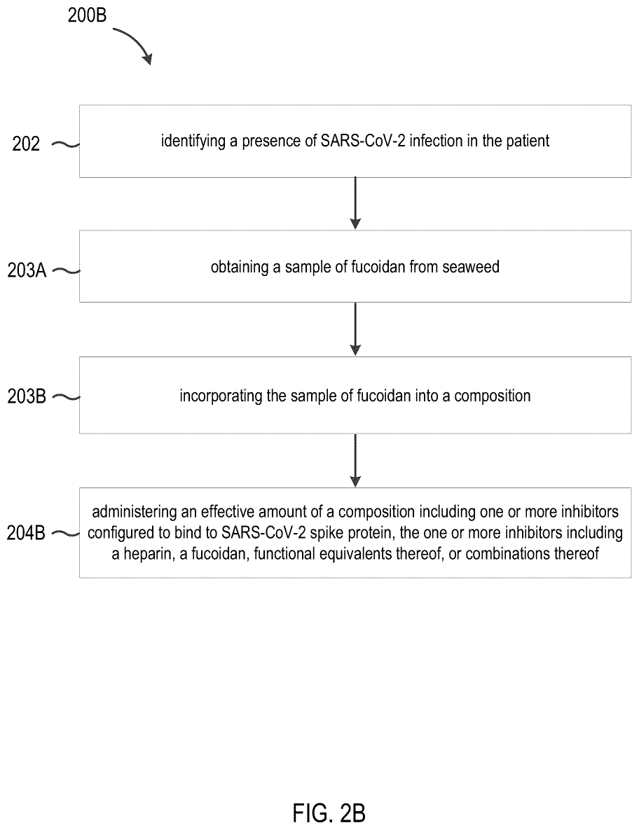

Referring now to FIG. 2A, some embodiments of the present disclosure are directed to a method 200A of inhibiting SARS-COV-2 in a patient. At 202, the presence of SARS-COV-2 infection is identified in the patient. In some embodiments, the SARS-COV-2 infection is identified by any suitable test. At 204A, an effective amount of a composition including one or more inhibitors configured to bind to SARS-COV-2 spike protein is administered to the patient. In some embodiments, the composition includes an amount of crude extracts from a seaweed (or kelp) including a concentration of sulfated polysaccharides. In some embodiments, the effective amount of composition results in a peak plasma concentration of inhibitor in the patient less than about 60 μM. In some embodiments, the effective amount of composition results in a peak plasma concentration of inhibitor in the patient less than about 10 μM. In some embodiments, the effective amount of composition results in a peak plasma concentration of inhibitor in the patient less than about 5 μM. In some embodiments, the effective amount of composition results in a peak plasma concentration of inhibitor in the patient less than about 2 μM. In some embodiments, the effective amount of composition results in a peak plasma concentration of inhibitor in the patient less than about 1 μM.

In some embodiments, the composition is administered by any suitable drug delivery process, e.g., orally, nasally, via inhalation, nebulization, transdermally, intravenously, or combinations thereof. In some embodiments, the composition is administered orally, nasally, pulmonary, transdermally, or combinations thereof. Current standard therapeutics for patients with a SARS-COV-2 infection, e.g., remdesivir, must be administered intravenously, so the ability of the compositions of the present disclosure to be delivered by more patient-friendly processes such as oral and nasal administration represent is large advantage compared with current interventions. In some embodiments, the composition includes one or more additional active ingredients. In some embodiments, the composition includes one or more pharmaceutically acceptable adjuvants, diluents, excipients, carriers, or combinations thereof. In some embodiments, the composition is included in a nutraceutical. In some embodiments, the nutraceutical includes inhibitors derived from GRAS organisms (Generally Recognized as Safe). In some embodiments, the composition is included in a coating, e.g., a coating layer for face masks or other surfaces, tightly binding the virus and improving the effectiveness of the surface to inhibit infection, e.g., enhance the effectiveness of the mask to block transmission of the virus. In some embodiments, the composition is included in filter materials, e.g., in A/C and other HVAC in buildings.

As discussed above, in some embodiments, the one or more inhibitors including a heparin, a fucoidan, functional equivalents thereof, or combinations thereof. In some embodiments, the one of more inhibitors includes unfractionated USP-heparin, TriS heparin, NACH, or combinations thereof. In some embodiments, the fucoidan includes the structure according to Formula I:

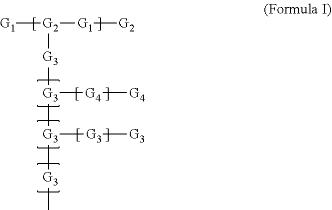

In some embodiments, G₁ is a glucuronic acid. In some embodiments, G₂ is a mannose. In some embodiments, G₃ is a fucose. In some embodiments, G₄ is a galactose, In some embodiments, a plurality of G₂, G₃, and G₄ are sulfated. In some embodiments, G₂, G₃, and G₄ are all fully sulfated. In some embodiments, the fucoidan includes the structure according to Formula II:

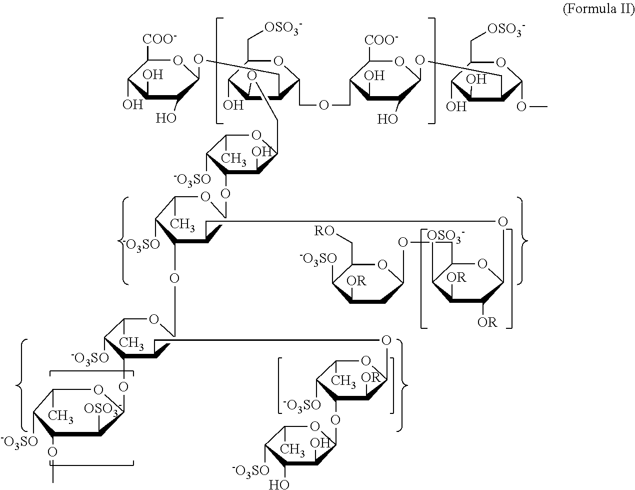

In some embodiments, each R is one of H or SO₃⁻. In some embodiments, the glycosaminoglycans in the composition have an average molecular weight of about 1 kDa, 5 kDa, 10 kDa, 15 kDa, 20 kDa, 25 kDa, 30 kDa, 35 kDa, 40 kDa, 45 kDa, 50 kDa, 60 kDa, 70 kDa, 80 kDa, 90 kDa, or 100 kDa. In some embodiments, the fucoidan has a molecular weight greater than about 10 kDa. In some embodiments, the fucoidan has a molecular weight of about 100 kDa. In some embodiments, the glycosaminoglycans are extracted from seaweed, e.g., Saccharina japonica. In some embodiments, the glycosaminoglycans are extracted from Saccharina latissima. In these embodiments, such as method 200B and referring now to FIG. 2B, at 203A, a sample of fucoidan is obtained from seaweed. In some embodiments, the sample is obtained by any suitable process. In some embodiments, the sample is substantially pure fucoidan. At 203B, the sample is then incorporated into the composition. At 204B, an effective amount of the composition is then administered to the patient, applied as a coating, e.g., to a mask or other filter, etc.

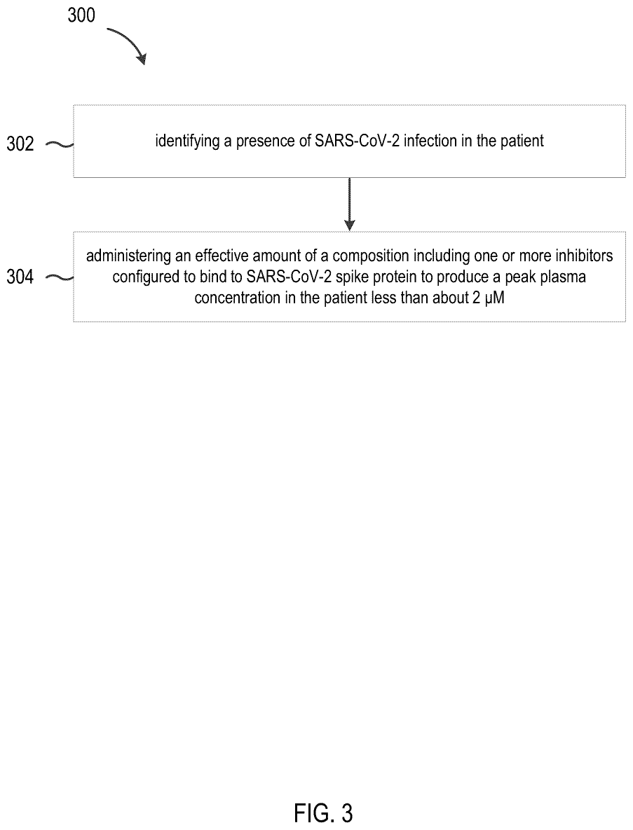

Referring now to FIG. 3, some embodiments of the present disclosure are directed to a method 300 of inhibiting SARS-COV-2 in a patient. At 302, the presence of SARS-COV-2 infection in the patient is identified. As discussed above, the diagnosis of a SARS-COV-2 can be facilitated via any suitable process. At 304, an effective amount of a composition including one or more inhibitors configured to bind to SARS-COV-2 spike protein is administered to the patient to produce a peak plasma concentration of inhibitor in the patient less than about 2 μM. The compositions suitable for use in the method 300 are those consistent with the embodiments identified above.

In an exemplary embodiment, the in vitro antiviral properties of heparin and other closely related polysaccharides were evaluated to assess the relevance of heparin-related GAGs and other sulfated polysaccharides as part of the pharmacopeia of potential therapeutics that target SARS-COV-2. Vero-CCL81, which expresses both ACE2 and TMPRSS2, were used for viral replication at high titer for use in antiviral assays.

Preparation of heparin SPR biochip. Spike proteins: “SARS-COV2” (MW: 101.7 kDa, Cat: 40591-V02H), coronavirus spike; “SARS-COV” (MW: 74.4 kDa, Cat: 40150-V08B1); and “MERS-COV” (MW: 142.2 kDa, Cat: 40069-V08B) were purchased from Sino Biological Inc. Porcine intestinal mucosa heparin (MW ˜17 kDa) was from Celsus Laboratories, Inc. TriS-heparin, MW ˜17.6 kDa, was chemoenzymatically synthesized. NACH, MW ˜4 kDa, was chemically synthesized from enoxaparin (Sanofi-Aventis). RPI-27, MW ˜100 kDa, and RPI-28, MW ˜12 kDa were extracted from seaweed. Streptavidin (SA) sensor chips were from GE Healthcare (Uppsala, Sweden). SPR measurements were performed on a BIAcore 3000 operated using BIAcore 3000 control and BIAevaluation software (version 4.0.1).

Biotinylated heparin was prepared by conjugating its reducing end to amine-PEG3-Biotin (Pierce, Rockford, IL). In brief, heparin (2 mg) and amine-PEG3-Biotin (2 mg, Pierce, Rockford, IL) were dissolved in 200 μL H₂O, 10 mg NaCNBH₃ was added. The reaction mixture was heated at 70° C. for 24 h, after that a further 10 mg NaCNBH₃ was added and the reaction was heated at 70° C. for another 24 h. After cooling to room temperature, the mixture was desalted with the spin column (3,000 MWCO). Biotinylated heparin was collected, freeze-dried and used for SA chip preparation.

The biotinylated heparin was immobilized to the SA chip based on the manufacturer's protocol. The successful immobilization of heparin was confirmed by the observation of a 600-resonance unit (RU) increase on the sensor chip. The control flow cell (FC1) was prepared by 2 min injection with saturated biotin.

The protein samples were diluted in HBS-EP buffer (0.01 M HEPES, 0.15 M NaCl, 3 mM EDTA, 0.005% surfactant P20, pH 7.4). Different dilutions of protein samples were injected at a flow rate of 30 μL/min. At the end of the sample injection, the same buffer was flowed over the sensor surface to facilitate dissociation. After a 3 min dissociation time, the sensor surface was regenerated by injecting with 30 μL of 0.25% sodium dodecyl sulfate (SDS) to get fully regenerated surface. The response was monitored as a function of time (sensorgram) at 25° C.

Solution competition study between heparin on chip surface and heparin, trisulfated heparin analog, chemically modified heparin or GAGs in solution using SPR. Spike protein (50 nM) mixed with 1 μM of heparin, TriS-heparin, NACH, RPI-27, and RPI-28 in HBS-EP buffer were injected over a chip, on which heparin was immobilized, at a flow rate of 30 μL/min, respectively. After each run, the dissociation and the regeneration were performed as described above.

Virus isolation and propagation. SARS-COV-2 isolated from a Korean patient was provided by the National Culture Collection for Pathogens (NCCP43326). The isolate was propagated in Vero-CCL81 cells (Korean Cell Line Bank, KCLB No. 10081, Korea) with Dulbecco's modified Eagle's medium (DMEM), supplemented with 2% FBS, 50 U/ml penicillin, and 50 μg/mL streptomycin. Three days post virus infection, culture supernatants were collected, aliquoted, and stored at-80° C. Viral titer was determined by focus formation assay. Vero cells (2×104 cells/well, 100 μL/well) were infected with a 10-fold serial dilution of SARS-CoV-2 for 1 h and then removed inoculum. Next, 2% carboxymethyl cellulose in 4% FBS in DMEM were overlaid to the each well. Subsequent procedures were conducted under the identical conditions as described below in a focus reduction assay. The average of six wells was calculated as focus forming units (FFU) per ml. All the procedures were performed in a biosafety level 3 laboratory.

Cytotoxicity assay. Water Soluble Tetrazolium Salt-1 (WST-1) assay (Takara Bio Inc., Japan) was performed following the manufacturer's protocol. Briefly, Vero cells (2×10⁴ cells/well, 100 μL/well) were seeded in flat-bottom 96-well plates and incubated overnight at 37° C. to evaluate the cytotoxicity of polysaccharides. Cells were treated with a different dose of each polysaccharide (0.5, 0.05, 0.005 mg/ml). After 48 h incubation at 37° C., 10 μL premix WST-1 was added per well and incubated for 1 h at 37° C. Absorbance was measured at 430 nm using ELISA plate reader (Epoch, Bio-Tek Instruments, Inc., USA). The 50% cytotoxic concentration for inhibitors (EC₅₀; polysaccharide concentration that reduced the cell viability by 50% compared to the cell only control) was determined using nonlinear regression analysis (GraphPad Prism 8.4, USA). Each experiment was performed in duplicate wells and repeated three times.

Focus reduction assay. Vero cells (2×10⁴ cells/well, 100 μL/well) were seeded in 96 well plates to confirm antiviral activity of the polysaccharides. SARS-COV-2 (multiplicity of infection; MOI=2.5×10⁻³) was pre-incubated with several doses of the polysaccharide (0.5, 0.05, and 0.005 mg/L for 1 h at 37° C.). Each mixture of virus and polysaccharide was then added to prepared cells to allow infection for 1 h. These virus-polysaccharide mixtures were removed and 2% carboxymethyl cellulose in 4% FBS in DMEM were overlaid to the infected cells. The plates were further cultured at 37° C. for 2 days. After incubation, the cells were washed with cold PBS, fixed with 4% paraformaldehyde phosphate buffer solution for 30 min at 4° C., and permeabilized with 0.5% Triton X-100 for 20 min at room temperature. After washing, the cells were incubated with SARS-COV-2 spike antibody (1:10000, Sino Bio Inc.) for 45 min at room temperature. The plates were then washed with 0.05% Tween 20 and then incubated with HRP-conjugated goat rabbit (1:10000, Abcam) for 45 min at room temperature. After repeating the washing, and the cells were stained with 50 μL True Blue peroxidase substrate (KPL) for 20 min in the dark. Subsequently, the plates were washed with tap water and were dried completely. Plate images were captured using an Immunospot CTL reader (S6 Universal analyzer) and the number of foci/well counted. SARS-COV-2 was tested against each polysaccharide concentration in quadruplicate wells. Experiments were repeated three times. The 50% inhibitory concentration of each polysaccharide against SARS-COV-2 (IC₅₀; polysaccharide concentration that inhibited FFU formation by 50% compared to the cell only control) was determined using non-linear regression analysis (GraphPad Prism 8.4, USA).

Heparin docking model. The crystal structure (PDB ID: 6VW1) of the molecule that included the RBD (receptor binding domain) of the SARS-COV-2 spike protein bound to the angiotensin-converting enzyme 2 (ACE2) receptor has been solved. Without wishing to be bound by theory, the availability of this structure allowed generation of a model descriptive of the mechanism through which glycosaminoglycans may inhibit spike protein RBD-ACE2 binding. The RBD-ACE2 binding interface is stabilized by an extensive hydrogen bonding network involving sidechains of several residues on both RBD and ACE2. The identity of the residues that defined this RBD-ACE2 binding interface were used to perform a docking experiment to assess the ability of a heparin octasaccharides (octasaccharide taken from PDB 5UE2) to inhibit the interaction between RBD and ACE2. It was hypothesized that the available polar sidechains of N487, Y489, Q493, Q498 and Y505 on the spike protein RBD along with other residues would bind to heparin and inhibit RBD-ACE2 interaction. Using the autodock software suite a search was performed of various conformations that the glycosaminoglycan could sample at the ACE2 binding surface of the spike protein RBD. From this experiment, a binding mode of the heparin octasaccharide was identified that would restrict RBD-ACE2 binding. In this conformation, the heparin octasaccharide forms a hydrogen bond network with N448, N450, Q493 and N501 that aids in its occupancy of this binding region and sterically restricts access to Q498, Y489 and Y505.

In a further exemplary embodiment, RPI-27 and RPI-28 fucoidan were prepared from the brown algae S. japonica. In this exemplary embodiment, RPI-28 was derived from RPI-27 by treating a 1.5% (w/v) aqueous solution of RPI-27 with hydrogen peroxide and ascorbic acid, both at a final concentration of 25 mM, for 2 h at room temperature.

Additionally, a crude fucoidan extract was isolated from the brown algae S. latissima provided as ground kelp by the Montauk Seaweed Supply Co. (Montauk, NY). Crude extracts from S. latissima were generated as follows: the dried kelp was ground and suspended in 10 times its weight of distilled water. The suspension was thoroughly stirred and heated in a water bath at 70° C. for 2 h. After centrifugation at 6,000×g, the supernatant was collected and ethanol was added to the solution at a final concentration of 30% (v/v) ethanol, and the resulting precipitate was removed by centrifugation at 6,000×g. The supernatant was then mixed with ethanol to achieve a final concentration of 70% (v/v) ethanol, and the resulting precipitate was collected by centrifugation at 6,000×g and labeled as the crude extract from S. latissima.

The crude extract from S. latissima, as well as the fucoidans RPI-27 and RPI-28, were subjected to a sequential fractionation procedure. To this end, the crude extract and RPI-27 and RPI-28 were dissolved in distilled water and subjected to sequential MWCO separation and strong anion-exchange (SAX) fractionation. For the former, the glycan solution was sequentially passed through a series of ultrafiltration steps using 100, 30, 10, and 3 kDa filters. Each fraction was collected, freeze-dried, and weighed to calculate isolated yield. Fractions with a recovery of over 14% were further fractionated using SAX chromatography. Twenty milligrams of the major fractions were dissolved in 0.2 M NaCl and loaded onto a Vivapure D Maxi H column. Elution was performed sequentially using 0.5, 1.0, 2.0, and 5.0 M NaCl, sequentially. Each subfraction was collected, desalted via ultrafiltration using 3 kDa filters, and freeze-dried.

The overall weight recovery of RPI-27, RPI-28, and algal crude extract after MWCO fractionation was 74-88%. The resulting major fractions were further subjected to SAX fractionation, with a further weight recovery of 68-95%. RPI-27 comprised predominantly high-molecular-weight polysaccharides, with Mn=147 kDa and polydispersity index (PDI, Mw/Mn)=1.19. Following MWCO fractionation the >100 kDa fraction was nearly 5-fold in excess of the 30-100 kDa fraction. Relatively high salt was used to elute the two major RPI-27 fractions from the SAX column; the higher molecular weight fraction using 5 M aq. NaCl for greater elution than the lower molecular weight fraction. Four major subfractions were obtained from SAX separation and used in further studies: RPI-27>100 kDa (2 M); RPI-27>100 kDa (5 M); RPI-27 30-100 kDa (2 M); and RPI-27 30-100 kDa (5 M). RPI-28 yielded three major MWCO fractions: 30-100 kDa, 10-30 kDa, and 3-10 kDa, with over half <30 kDa. Six subfractions were isolated and used in further assays: RPI-28 30-100 kDa (1 M); RPI-28 30-100 kDa (2 M); RPI-28 10-30 kDa (1 M); RPI-28 10-30 kDa (2 M); RPI-28 3-10 kDa (1 M); and RPI-28 3-10 kDa (2 M).

The crude polysaccharide extract from S. latissima included fucoidan (FSl ) with a relatively high proportion of small polysaccharide chains of Mn 33.7 kDa. These MWCO fractions were somewhat intermediate to those of RPI-27 and RPI-28, made up of roughly evenly split fractions of >100 kDa, 30-100 kDa, and <30 kDa. Based on SAX analysis, the 30-100kDa fraction contained approximately 60% of the component unbound to the SAX column, likely indicating the presence of laminarin in the crude extract. The subfractions selected for further investigation from FSl were: FSl>100 kDa (1 M) and FSl>100 kDa (2 M). These subfractions of FSl had quite high molecular weights resulting from the removal of a large fraction of the low molecular weight chains that had been present in the crude FSl extract. The crude extract and MWCO/SAX fractions from S. latissima also contained a lower sulfate content than RPI-27 and RPI-28. Moreover, the crude extract contained a significant amount of glucose, likely derived from laminarin. The monosaccharide composition of the subfractions from S. latissima was complex, with a higher abundance of mannose, galactose, xylose, glucuronic acid, and a small amount of glucosamine

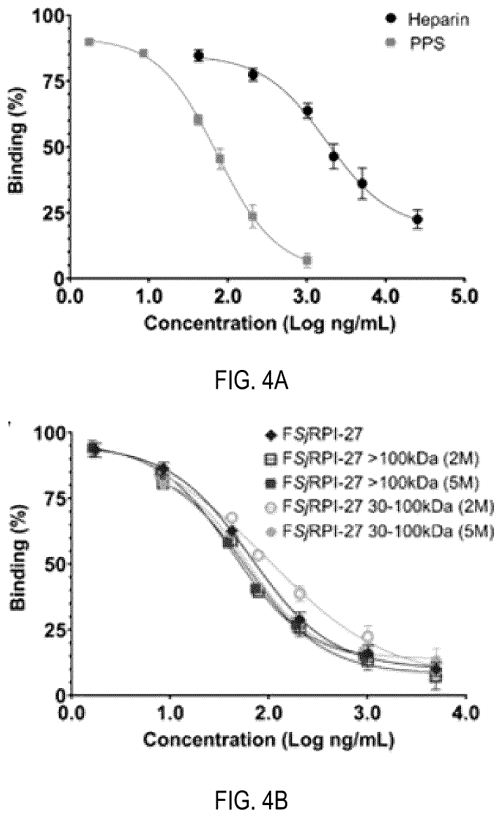

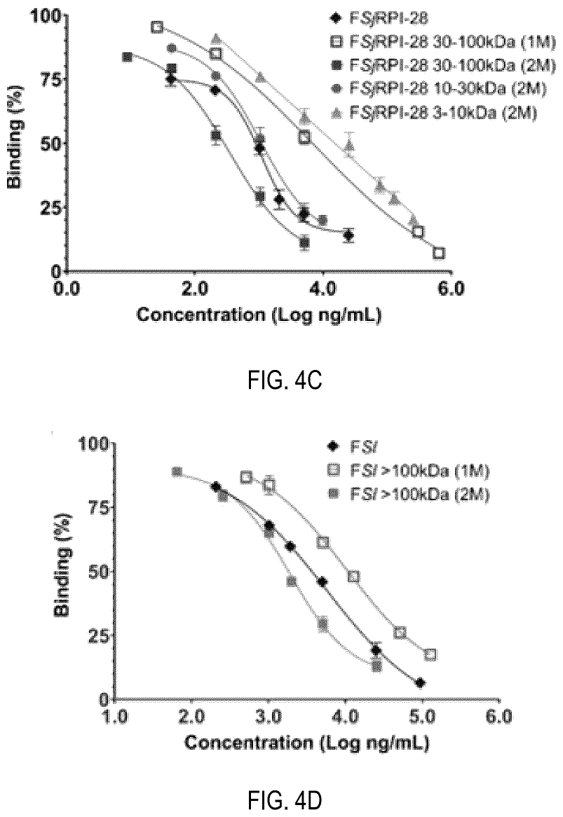

Referring now to FIGS. 4A-4D, dose response curves in SPR solution competition analysis for each algal subfraction, along with crude or unfractionated materials, were prepared, along with IC₅₀ values. Dose response curves for heparin and PPS are shown for comparison in FIG. 4A, giving rise to IC₅₀ values of 1.8 μg/mL and 74 ng/ml, respectively.

The RPI-27 series (see FIG. 4B) exhibited significant competitive inhibition activity against SGP binding to heparin, with an IC₅₀=43.4 ng/ml for the >100 kDa, 5 M NaCl subfraction. The RPI-28 series (see FIG. 4C) and subfractions from S. latissima (see FIG. 4D) were also potent in blocking SGP RBD-heparin binding, e.g., RPI-28 30-100 kDa, 2 M NaCl with IC₅₀=341 ng/mL.

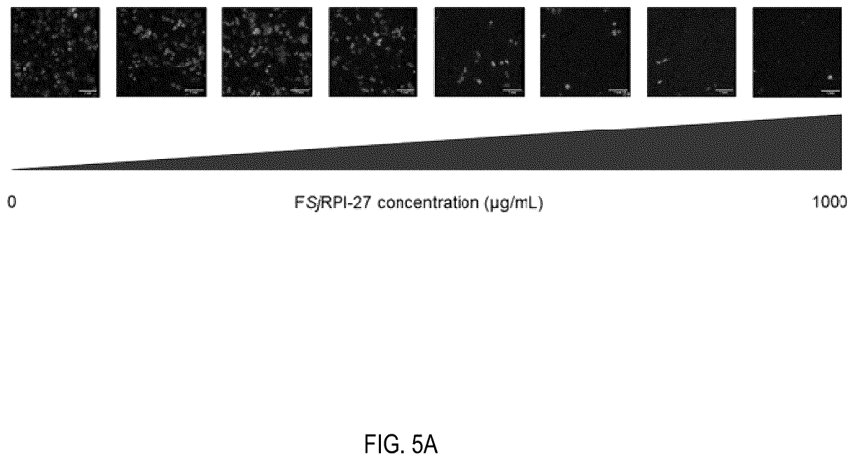

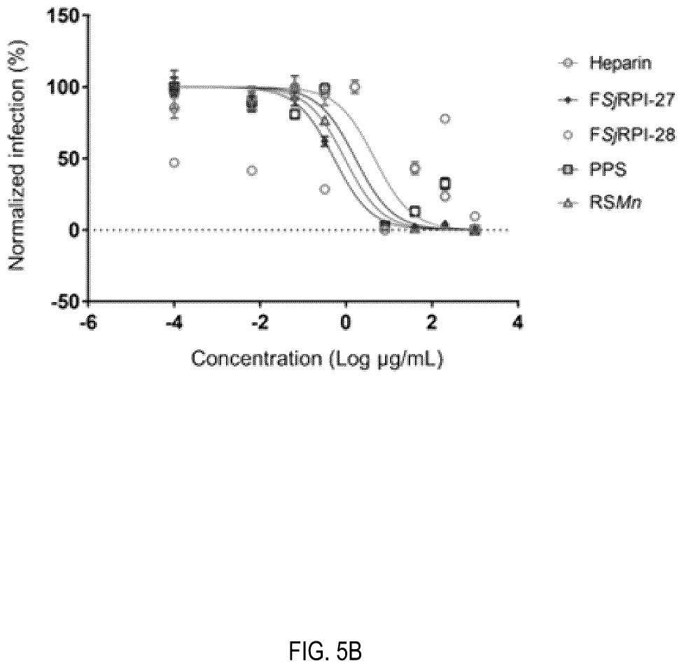

Referring now to FIGS. 5A-5B, the effect of a subset of the fractions/subfractions, including unfractionated RPI-27 and RPI-28, >100 kDa, 5 M NaCl RPI-27, on inhibition of cell-based viral infection was examined. Pseudovirus particles containing surface Wuhan-Hu-1 SGP were prepared and infection of human ACE2 HEK-293T cells was assessed in 96-well plates. Inhibition of entry was evident via decreased fluorescence in each well (representative wells for each glycan tested) as a function of glycan concentration (see FIG. 5A). From the dose response curves (see FIG. 5B), IC₅₀ values of unfractionated RPI-27 was 0.52 μg/mL. These fractions were significantly more potent than PPS and heparin, which had ICso values of 1.6 and 4.6 μg/mL, respectively.

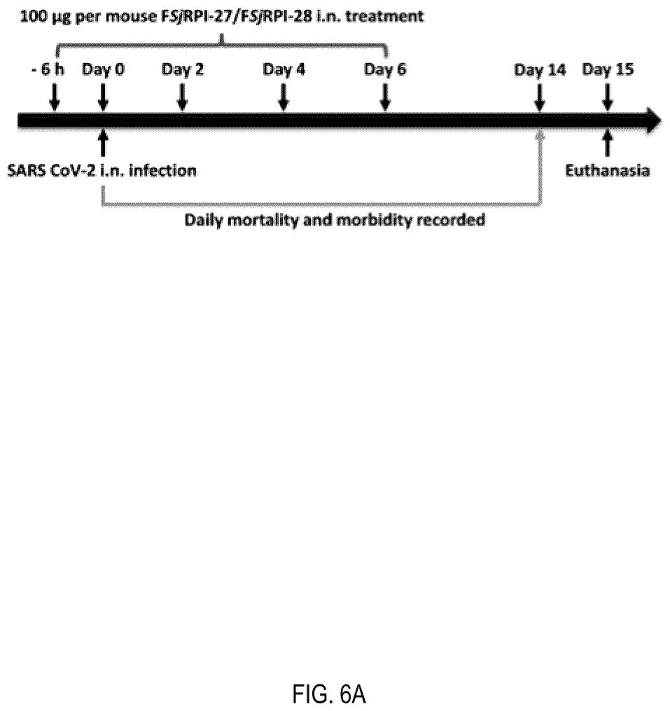

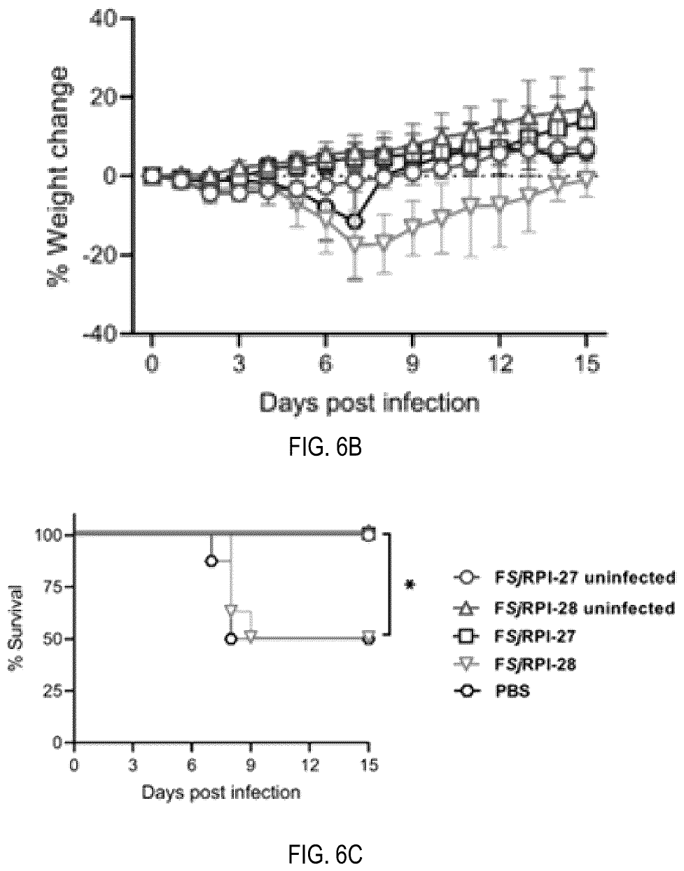

Referring now to FIG. 6A-6C, consistent with the in vitro study, i.n. treatment with RPI-27 before and during an LD₅₀ SARS-COV-2 infection of hACE2 transgenic mice led to significant protection, with 100% survival and no noticeable morbidity, as shown by the absence of weight loss. Treatment with RPI-27 alone in the absence of infection likewise led to no changes in weight, indicating a lack of fucoidan toxicity.

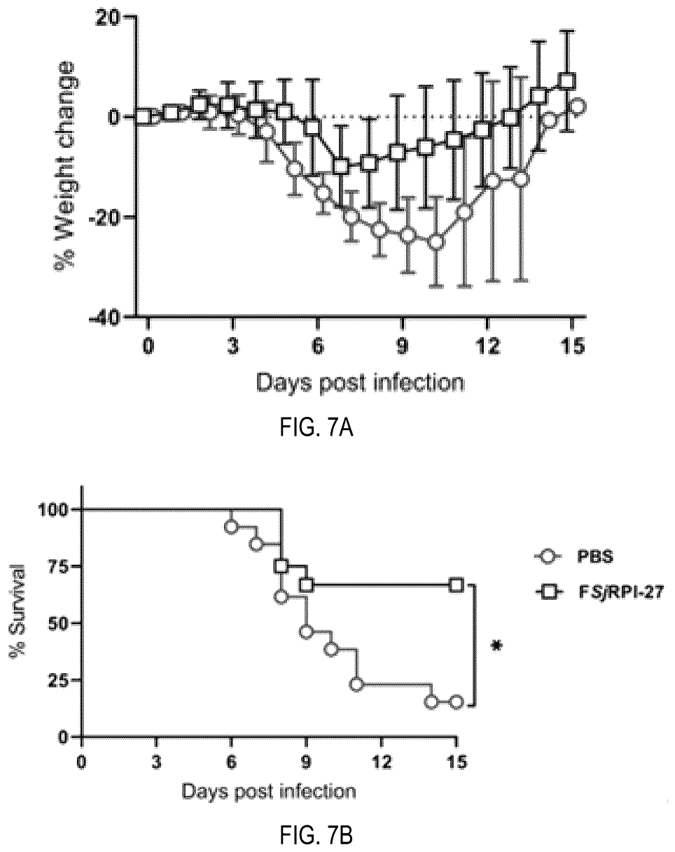

Referring now to FIGS. 7A-7B, the protective efficacy of RPI-27 was further evaluated using a five-fold greater SARS COV-2 infection dose. In this case, 85% of PBS-treated mice succumbed to infection with substantial weigh loss (20-35%) following challenge with SARS COV-2 virus. In contrast, approximately two-thirds of the RPI-27 treated mice survived and regained their body weight starting on day 7 post-infection. At 15 dpi, none of the surviving mice showed any apparent signs of disease.

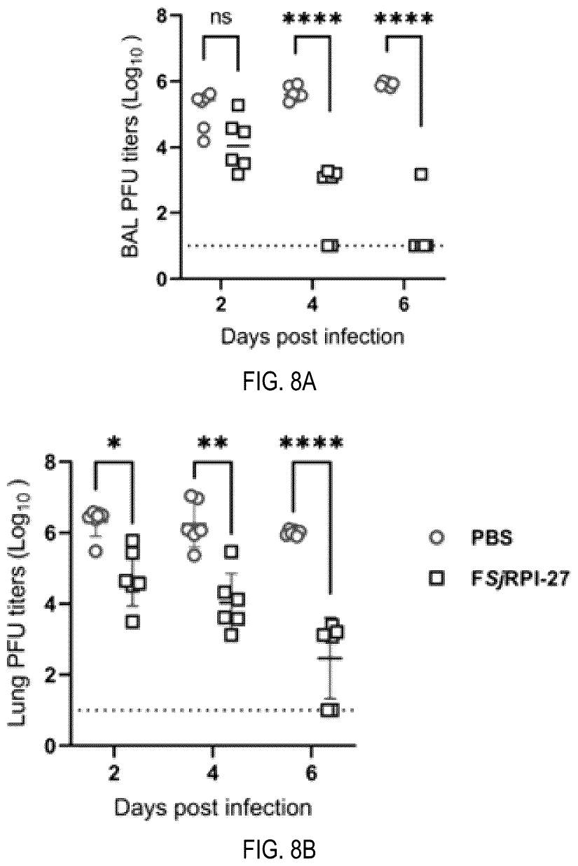

Referring now to FIG. 8A-8B, viral loads in bronchoalveolar lavages (BAL) and lung homogenates from animals infected with the higher dose of SARS-COV-2 were measured to determine whether RPI-27 would prevent infection and/or enhance viral clearance. Compared to day 0, PBS-treated infected mice demonstrated a 2-3 log₁₀ increase in BAL and lung viral titers at all timepoints. Conversely, on day 2 post challenge, BAL of RPI-27 treated mice had a moderate (1.5 log₁₀) reduction in viral titers compared to PBS-treated infected mice (see FIG. 8A). At 4 dpi, the BAL viral titers were further reduced to 2.67 logo after RPI-27 treatment, and by 6 dpi, only one RPI-27-treated mouse still contained detectable virus. Lung homogenates showed a 1 and 2 log₁₀ reduction in viral burden at 2 and 4 dpi, respectively, after RPI-27 treatment compared to PBS treatment (see FIG. 8B). By 6 dpi, the mean lung homogenate viral titers in the RPI-27 treated group was 2.13 log₁₀ with no replicating virus detected in more than 33% of the surviving mice. Taken together, the results indicate that RPI-27 treatment reduces initial viral burden and also enhances viral clearance during the course of infection.

Methods and systems of the present disclosure are advantageous to provide compositions showing antiviral activity and low cytotoxicity, and thus promising for clinical use against severe acute respiratory syndrome coronavirus 2. Along these lines, SARS-COV-2 has been found to infect a wide range of tissues that possess sufficient ACE2 levels, including the nose and the gastrointestinal tract. Potential routes of delivery of the non-anticoagulant polysaccharide inhibitors in these compositions, including the fucoidans (RPI-27, and RPI-28) and the TriS-heparin, could include a nasal spray, metered dose inhaler, or oral delivery. This is beneficial compared with remdesivir, which is delivered intravenously. Indeed, when taken orally, the fucoidans, isolated from edible sulfated seaweed polysaccharides, are considered as “Generally Recognized as Safe” and heparin, an approved drug, is not orally bioavailable. Interestingly, a retrospective clinical study suggests that the administration of anticoagulants, such as heparin, may provide better outcomes for patients hospitalized with COVID-19, including a dramatic reduction in mortality of intubated patients. Inhaled heparin has additional benefits such as reducing pulmonary coagulopathy and inflammation without producing systemic bleeding. To this end, treatment with fucoidans, nebulized heparin, TriS-heparin, etc., in combination with or without current antiviral therapies, should be assessed in human patients suffering from COVID-19. The interactions between this class of compounds and viral spike proteins also suggest efficacy with common cold coronaviruses, which comprise 25-30% of all common cold infections. The polysaccharides would be expected to be very cheap and not much would be needed to trap virus particles.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A composition for inhibiting severe acute respiratory syndrome coronavirus 2 (SARS-CoV-2), comprising:
a plurality of inhibitors configured to bind to SARS-COV-2 spike protein,
wherein at least one of the inhibitors includes a heparin, a fucoidan, or combinations thereof.

2. The composition according to claim 1, wherein the inhibitors include unfractionated USP-heparin, a trisulfated (TriS) heparin, a non-anticoagulant low molecular weight heparin (NACH), or combinations thereof.

3. The composition according to claim 1, wherein the inhibitors include a fucoidan, wherein the fucoidan includes the structure according to Formula I: 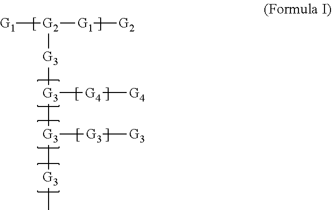
wherein G₁ is a glucuronic acid; G₂ is a mannose; G₃ is a fucose; and G₄ is a galactose.

4. The composition according to claim 3, wherein a plurality of G₂, G₃, and G₄ are sulfated.

5. The composition according to claim 3, wherein the fucoidan includes the structure according to Formula II: 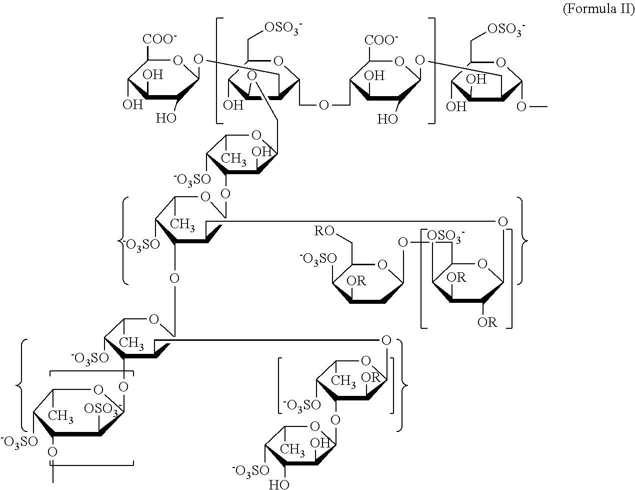
wherein each R is one of H or SO₃⁻.

6. The composition according to claim 5, wherein the fucoidan has a molecular weight greater than about 10 kDa.

7. The composition according to claim 6, wherein the fucoidan has a molecular weight of about 100 kDa.

8. The composition according to claim 1, wherein the composition includes:
one or more additional active ingredients; or
one or more pharmaceutically acceptable adjuvants, diluents, excipients, carriers, or combinations thereof.

9. A method of inhibiting severe acute respiratory syndrome coronavirus 2 (SARS-COV-2) in a patient, comprising:
identifying a presence of SARS-COV-2 infection in the patient; and
administering to the patient an effective amount of a composition including one or more inhibitors configured to bind to SARS-COV-2 spike protein, the one or more inhibitors including a heparin, a fucoidan, functional equivalents thereof, or combinations thereof.

10. The method according to claim 9, wherein the one of more inhibitors includes unfractionated USP-heparin, a trisulfated (TriS) heparin, a non-anticoagulant low molecular weight heparin (NACH), or combinations thereof.

11. The method according to claim 9, wherein the fucoidan includes the structure according to Formula I: 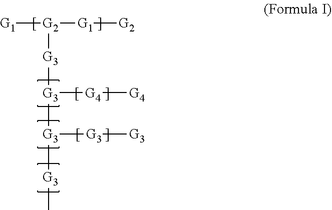
wherein G₁ is a glucuronic acid; G₂ is a mannose; G₃ is a fucose; and G₄ is a galactose, wherein a plurality of G₂, G₃, and G₄ are sulfated.

12. The method according to claim 11, wherein the fucoidan includes the structure according to Formula II: 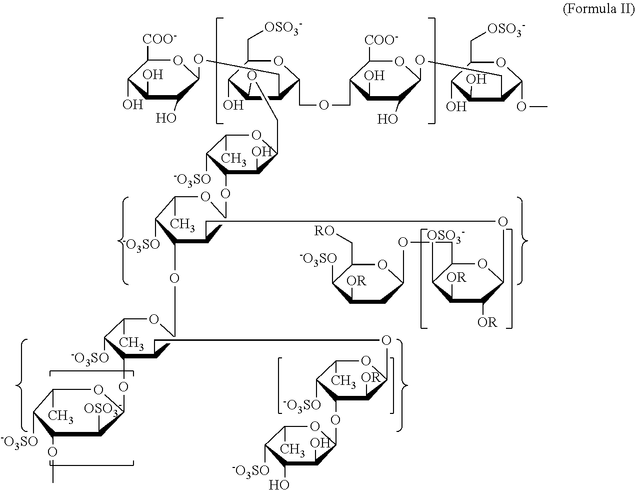
wherein each R is one of H or SO₃⁻.

13. The method according to claim 12, wherein the fucoidan has a molecular weight greater than about 10 kDa.

14. The method according to claim 13, wherein the fucoidan has a molecular weight of about 100 kDa.

15. The method according to claim 9, wherein administering the effective amount of the composition produces a peak plasma concentration in the patient less than about 60 μM.

16. The method according to claim 15, wherein administering the effective amount of the composition produces a peak plasma concentration in the patient less than about 5 μM.

17. The method according to claim 9, wherein the composition is administered orally, nasally, pulmonary, or combinations thereof.

18. The method according to claim 9, further comprising:
obtaining a sample of fucoidan from seaweed; and
incorporating the sample of fucoidan into a composition.

19. A method of inhibiting severe acute respiratory syndrome coronavirus 2 (SARS-COV-2) in a patient, comprising:
identifying a presence of SARS-COV-2 infection in the patient; and
administering to the patient an effective amount of a composition including one or more inhibitors configured to bind to SARS-COV-2 spike protein to produce a peak plasma concentration in the patient less than about 2 μM, the one or more inhibitors including the structure according to Formula I: 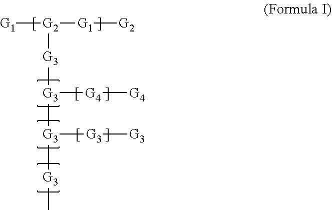
wherein G₁ is a glucuronic acid; G₂ is a mannose; G₃ is a fucose; and G₄ is a galactose, wherein a plurality of G₂, G₃, and G₄ are sulfated.

20. The method according to claim 19, wherein the one or more inhibitors includes the structure according to Formula II: 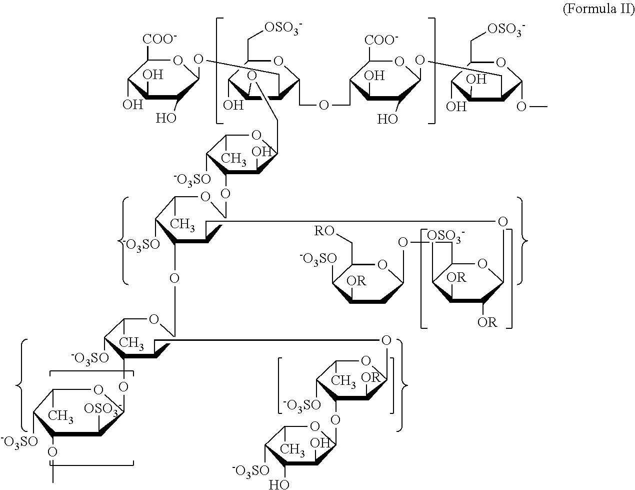
wherein each R is one of H or SO₃⁻.
